(19) United States
(12) Patent Application Publication
(10) Pub. No.: US 2023/0391762 A1
(43) Pub. Date: Dec. 7, 2023

(54) A PROCESS FOR PREPARATION OF PYROXASULFONE

(71) Applicant: UPL Limited, Mumbai (IN)

(72) Inventors: Prashant Vasant Kini, Mumbai (IN); Sopan Nagnath Gandhale, Mumbai (IN); Debasish Sengupta, Mumbai (IN); Sandip Sahebrao Gulve, Mumbai (IN); Shrikant Muqutrao Anpat, Mumbai (IN); Manish Madhukar Borse, Mumbai (IN)

(21) Appl. No.: 18/043,431

(22) PCT Filed: Aug. 12, 2022
(86) PCT No.: PCT/IN2022/050724

§ 371 Date: Feb. 28, 2023

(30) Foreign Application Priority Data
Aug. 12, 2021 (IN) 202121036558

(51) Int. Cl. C07D 413/12; A01N 43/80; A01P 13/00

(52) U.S. Cl. CPC C07D 413/12; A01N 43/80; A01P 13/00

(57) ABSTRACT

The present invention relates to a process for preparation of Pyroxasulfone substantially free of undesired impurity, by oxidizing a compound of formula (II) at a temperature ranging from 40 to 80° C.

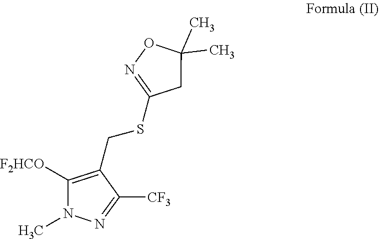

### FIELD OF THE INVENTION

The present invention relates to a process for preparation of Pyroxasulfone substantially free of undesired impurity.

### BACKGROUND OF THE INVENTION

Pyroxasulfone is an herbicide belonging to the group of pyrazolium. Pyroxasulfone is chemically known as 3-[5-(difluoromethoxy)-1-methyl-3-(trifluoromethyl)pyrazol-4-ylmethylsulfonyl]-4,5-dihydro-5,5-dimethyl-1,2-oxazole and represented by compound of formula (I).

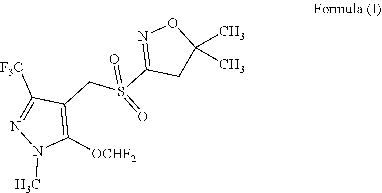 [SMILES: Cn1nc(C(F)(F)F)c(CS(=O)(=O)C2=NOC(C)(C)C2)c1OC(F)F]

Pyroxasulfone is a pre-emergence herbicide that inhibits the biosynthesis of very long chain fatty acids. It can be used to effectively control grass and broad-leaved weeds in corn, soybean and wheat fields.

Pyroxasulfone was first disclosed in U.S. Pat. No. 7,238,689.

Currently, few processes for the preparation of Pyroxasulfone are known.

One of such method is described in US20120264947 which involves the oxidation of compound of formula (II) in presence of sodium tungstate dihydrate, hydrogen peroxide and acetic acid at room temperature for 16 hours. The reaction can be represented as follows

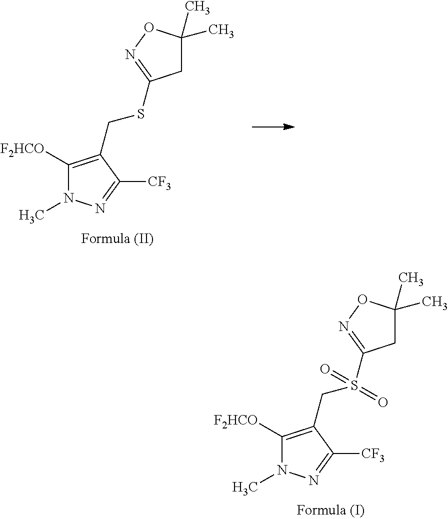

Unfortunately, the yield of the Pyroxasulfone obtained using this process is about 30-35% which is very low and hence, the process is not acceptable at industrial scale.

Another drawback of said process is that it leads incomplete oxidation and generation of undesired monoxide compound of formula (III).

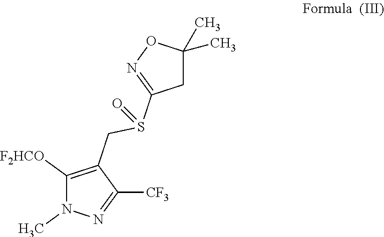 [SMILES: Cn1nc(C(F)(F)F)c(CS(=O)C2=NOC(C)(C)C2)c1OC(F)F]

This compound of formula (III) remains as a by-product in the final product i.e., Pyroxasulfone, and is very difficult to remove due to structural similarities. However, if compound of formula (III) is not separated, it may lead to quality deterioration of Pyroxasulfone formulation and there is also possibility of phytotoxicity to crops. Also, if such impurity is not removed/controlled, it would raise regulatory concerns. Hence, it is important to develop a process wherein oxidation reaction proceeds sufficiently and compound of formula (III) does not substantially remain in the product.

The present invention provides an improved process for the preparation of Pyroxasulfone which is substantially free of monoxide compound of formula (III), and wherein the yield and purity of final product is considerably increased.

### OBJECT OF THE INVENTION

It is an object of the present invention to provide a process for preparation of Pyroxasulfone of formula (I) substantially free of monoxide compound of formula (III).

Another object of the present invention is to provide a process for preparation of Pyroxasulfone of formula (I) in high yield.

Yet another object of the present invention is to provide a simple, cost effective and industrially viable process for preparation of Pyroxasulfone of formula (I).

### SUMMARY OF THE INVENTION

According to an aspect of the present invention, there is provided a process for preparation of Pyroxasulfone of formula (I) substantially free of monoxide compound of formula (III)

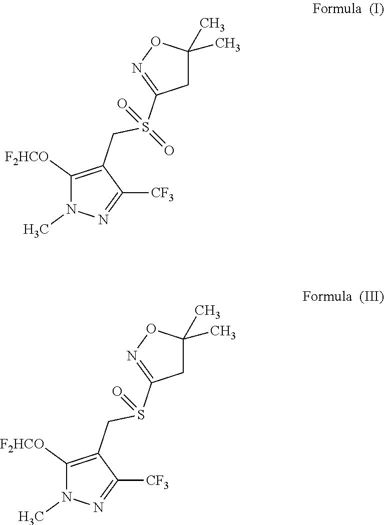 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

said process comprising:
oxidizing compound of formula (II) at a temperature ranging from 40 to 80° C.

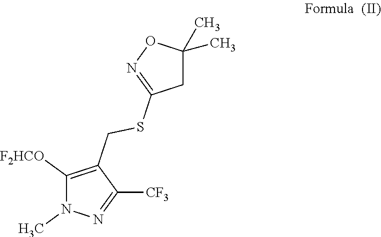 [SMILES: Cn1nc(C(F)(F)F)c(CSC2=NOC(C)(C)C2)c1OC(F)F]

According to an aspect of the present invention, there is provided a process for preparation of Pyroxasulfone of formula (I) substantially free of monoxide compound of formula (III)

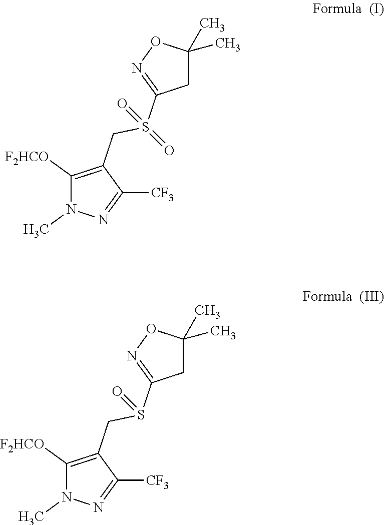 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

said process comprising:
oxidizing a compound of formula (II) in the presence of an oxidizing agent and a metal catalyst at a temperature ranging from 40 to 80° C. in acetic acid,

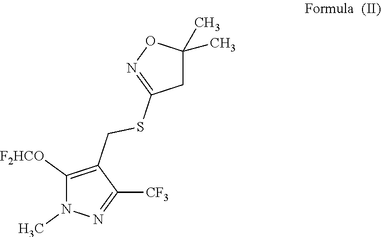 [SMILES: Cn1nc(C(F)(F)F)c(CSC2=NOC(C)(C)C2)c1OC(F)F]

wherein the amount of acetic acid used is in the range of 20 to 50 moles with respect to compound of formula (II).

### BRIEF DESCRIPTION OF DRAWING

FIG. 1 illustrates an HPLC chromatograph of example 1.

FIG. 2 illustrates an HPLC chromatograph of comparative example 1.

### DETAILED DESCRIPTION OF THE INVENTION

Those skilled in art will be aware that invention described herein is subject to variations and modifications other than those specifically described. It is to be understood that the invention described herein includes all such variations and modifications. The invention also includes all such steps, features, compositions and methods referred to or indicated in this specification, individually or collectively, and any and all combinations of any two or more said steps or features.

### Definitions

For convenience, before further description of the present invention, certain terms employed in the specification, examples are described here. These definitions should be read in light of the remainder of the disclosure and understood as by a person of skill in the art. Unless defined otherwise, all technical and scientific terms used herein have the same meaning as commonly understood by a person of ordinary skill in the art. The terms used throughout this specification are defined as follows, unless otherwise limited in specific instances.

The terms used herein are defined as follows.

As used in the specification and the claims, the singular forms “a”, “an”, and “the” include plural referents unless the context clearly dictates otherwise.

The term “about” or “approximately” as used herein is inclusive of the stated value and means within an acceptable range of deviation for particular value as determined by one of ordinary skill in the art, considering the measurement in question and the error associated with measurement of the particular quantity (i.e., the limitations of the measurement system). For example, “about” can mean within one or more standard deviations, or within ±10 or ±5 of the stated value. Recitation of ranges of values are merely intended to serve as a shorthand method of referring individually to each separate value falling within the range, unless otherwise indicated herein, and each separate value is incorporated into the 15 specification as if it were individually recited herein. The endpoints of all ranges are included within the range and independently combinable. It is understood that where a parameter range is provided, all integers within that range, and tenths thereof, are also provided. For example, “0.1-80%” includes 0.1%, 0.2%, 0.3%, etc. up to 80%.

As used herein, the terms “comprising” “including,” “having,” “containing,” “involving,” and the like are to be understood to be open-ended, i.e., to mean including but not limited to.

The terms “preferred” and “preferably” refer to embodiments of the invention that may afford certain benefits, under certain circumstances. In an embodiment, the aspects and embodiments described herein shall also be interpreted to replace the clause “comprising” with either “consisting of” or with “consisting essentially of” or with “consisting substantially of”.

The term “room temperature” unless stated otherwise, essentially means temperature in range of 20-35° C.

The term “purity” means purity as determined by HPLC (“High Pressure Liquid Chromatography”).

The term “Pyroxasulfone” as used herein, includes Pyroxasulfone free base or its salt and is used interchangeably throughout the disclosure.

The term “monoxide impurity” as used herein, includes “monoxide compound of formula (III)” and is used interchangeably throughout the disclosure.

As used herein, the term “substantially free of monoxide compound of formula (III)” refers to Pyroxasulfone containing less than or equal to 1% of monoxide compound of formula (III). Preferably, less than 0.5% w/w of monoxide compound of formula (III). More preferably less than 0.2% w/w of monoxide compound of formula (III).

The present disclosure is not to be limited in scope by the specific embodiments described herein, which are intended for the purposes of exemplification only.

According to an aspect of the present invention, there is provided a process for the preparation of Pyroxasulfone of formula (I) substantially free of monoxide compound of formula (III)

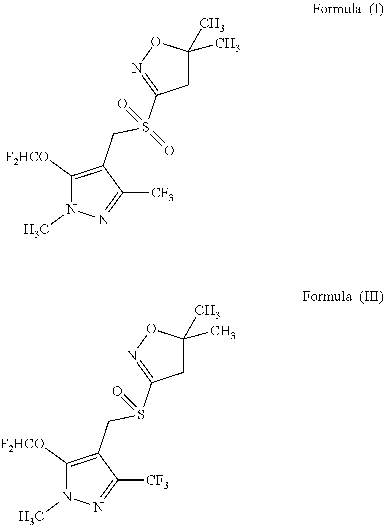 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

said process comprising:
oxidizing a compound of formula (II) at a temperature ranging from 40 to 80° C.

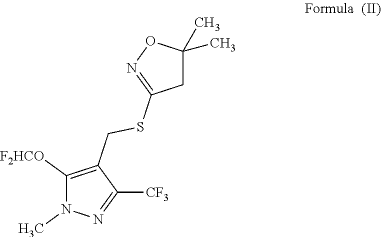 [SMILES: Cn1nc(C(F)(F)F)c(CSC2=NOC(C)(C)C2)c1OC(F)F]

In an embodiment, the compound of formula (II) is oxidized using an oxidizing agent.

In an embodiment, the oxidizing agent is selected from an organic peroxide or an inorganic peroxide.

In an embodiment, the oxidizing agent used is selected from, but not limited to, an organic peroxide such as m-chloroperbenzoic acid, performic acid, peracetic acid and the like; or an inorganic peroxides such as hydrogen peroxide, potassium permanganate, sodium periodate, potassium peroxymonosulfate (Oxone) and the like.

In an embodiment, the oxidizing agent used is hydrogen peroxide.

In an embodiment, the amount of oxidizing agent used is in the range of about 2.6 to 5 moles with respect to compound of formula (II)

In another embodiment, the compound of formula (II) is oxidised in the presence of a metal catalyst.

In an embodiment, the metal catalyst used is selected from, but not limited to, tungsten catalyst, molybdenum catalyst, titanium catalyst, zirconium catalyst or mixture thereof

Preferably, the catalyst used is tungsten catalyst tungsten, tungstic acid, tungstic acid salt, metallic tungsten, tungsten oxide, tungsten carbide or mixtures thereof.

More preferably, the tungsten catalyst used is sodium tungstate or its dihydrate.

In an embodiment, the amount of the metal catalyst used is in the range of 0.001 to 0.1 moles with respect to compound of formula (II).

In an embodiment, the compound of formula (II) is oxidized in an organic acid solvent. The organic acid solvent used is selected from, but not limited to, formic acid, acetic acid and the likes.

In an embodiment, said organic acid solvent is acetic acid.

The amount of said organic acid solvent used is in the range of 20 to 50 moles with respect to compound of formula (II).

In an embodiment, the compound of formula (II) is oxidized at temperature ranging from 40 to 80° C.

In an embodiment, the oxidation reaction is carried out for 1 to 12 hours.

In another embodiment, the oxidation reaction is carried out for a period in the range of 1 to 10 hours.

In yet another embodiment, the oxidation reaction is carried out for a period in the range of 1 to 8 hours.

According to another aspect of the present invention, there is provided a process for preparation of Pyroxasulfone of formula (I) substantially free of monoxide compound of formula (III)

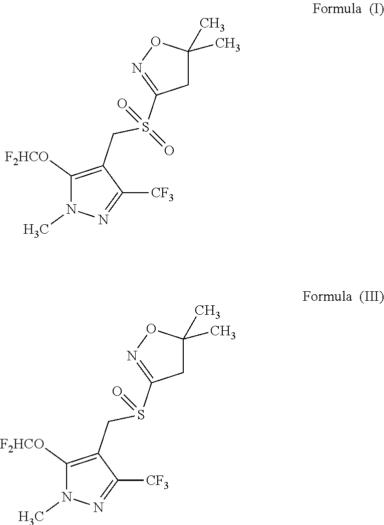 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

said process comprising:
oxidizing a compound of formula (II) in the presence of an oxidizing agent and a metal catalyst at a temperature ranging from 40 to 80° C. in acetic acid, and

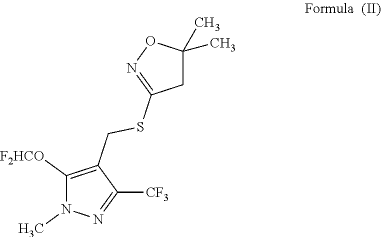 [SMILES: Cn1nc(C(F)(F)F)c(CSC2=NOC(C)(C)C2)c1OC(F)F]

wherein the amount of acetic acid used is in the range of 20 to 50 moles with respect to compound of formula (II).

In another embodiment, the oxidation is carried out at a temperature in the range of 40 to 70° C.

In yet another embodiment, the oxidation is carried out at a temperature in the range of 40 to 60° C.

In yet another embodiment, the oxidation is carried out at a temperature in the range of 40 to 50° C.

In an embodiment, the oxidizing agent used is selected from, but not limited to, an organic peroxide such as m-chloroperbenzoic acid, performic acid, peracetic acid and the like; or an inorganic peroxide such as hydrogen peroxide, potassium permanganate, sodium periodate and the like.

In an embodiment, the oxidizing agent used is hydrogen peroxide.

In an embodiment, the amount of oxidizing agent used is in the range of about 2.6 to 5 moles with respect to compound of formula (II).

In an embodiment, the oxidizing agent is added slowly at temperature ranging from 20 to 45° C. in 0.5 to 2 hours.

In an embodiment, the metal catalyst used is selected from, but not limited to, tungsten catalyst, molybdenum catalyst, titanium catalyst, zirconium catalyst or mixture thereof.

Preferably, the catalyst used is a tungsten catalyst selected from tungsten, tungstic acid, tungstic acid salt, metallic tungsten, tungsten oxide, tungsten carbide or mixtures thereof.

More preferably, the tungsten catalyst used is sodium tungstate or its dihydrate.

In an embodiment, the amount of the metal catalyst used is in the range of catalytic amount to 0.1 moles with respect to compound of formula (II).

In an embodiment, the process of the present invention comprises a step wherein the oxidation reaction is started at room temperature and then progressed to higher temperature ranging from 40 to 80° C.

In an embodiment, after completion of reaction water is added to reaction mixture to precipitate Pyroxasulfone.

In an embodiment, Pyroxasulfone obtained by present process is subjected to wash with water and an organic solvent; preferably with a non-polar solvent.

The non-polar solvent used is selected from, but not limited to, heptane, hexane, petroleum ether and the like.

In an embodiment, Pyroxasulfone is obtained in yield of more than 50%, preferably more than 70%.

In an embodiment the present invention provides Pyroxasulfone of formula (I) substantially free of monoxide compound of formula (III).

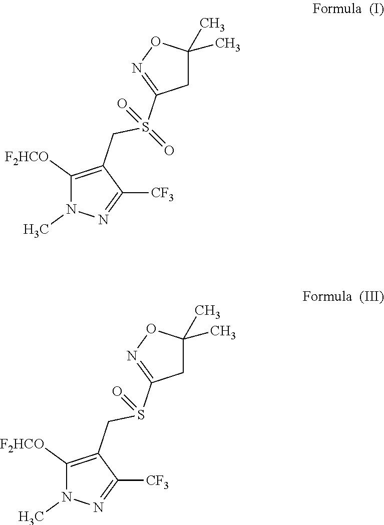 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

In an embodiment the present invention provides Pyroxasulfone of formula (I) having monoxide compound of formula (III) content less than or equal to 0.5% w/w.

In an embodiment the present invention provides Pyroxasulfone of formula (I) having monoxide compound of formula (III) content less than or equal to 0.2% w/w.

In a preferred embodiment the present invention provides Pyroxasulfone of formula (I) wherein monoxide compound of formula (III) is not detectable.

In an embodiment the present invention provides Pyroxasulfone of formula (I) having purity of more than 95%, preferably more than 98%.

In another embodiment there is provided use of Pyroxasulfone in the preparation of agrochemical composition or formulation.

In another embodiment there is provided use of Pyroxasulfone prepared using the process disclosed in the present invention in the preparation of agrochemical composition or formulation.

According to another embodiment, the present invention provides a herbicidal composition comprising pyroxasulfone prepared according to the process as described herein and at least one agrochemically acceptable excipients.

According to another embodiment, the present herbicide composition further comprising at least one additional herbicide.

According to another embodiment, the present herbicide composition further comprising additional herbicide. In an embodiment the additional herbicide is triazinone herbicide.

In an embodiment, the herbicidal composition comprising a combination of pyroxasulfone prepared according to the present process and a triazinone herbicide.

In an embodiment, the triazinone herbicide is selected from the group of ametridione, amibuzin, ethiozin, hexazinone, isomethiozin, metamitron, metribuzin, or trifludimoxazin. In an embodiment, the triazinone herbicide is metribuzin.

According to an embodiment, the present invention provides herbicidal composition comprising combination of pyroxasulfone prepared according to present process and metribuzin.

In an embodiment, the herbicide composition comprising pyroxasulfone prepared according to process as described herein; and at least one agriculturally acceptable excipient.

In an embodiment, agriculturally acceptable excipient/ carriers can be selected from one or more diluents, emulsifiers, fillers, anti-foaming agents, thickening agents, anti-freezing agents, freezing agents, a surfactant, a preservative, a coloring agent, a pH adjusting agent, dispersing agent, wetting agent and solvent. However, it should be appreciated that any other agriculturally acceptable excipients, as known to a person skilled in the art, may be used to serve its intended purpose. In an embodiment, the agriculturally acceptable excipients are present in an amount ranging from 0.01% to 90% by weight of the total composition.

Pyroxasulfone prepared according to present invention can be processed into an agricultural composition of various dosage forms by conventionally known methods.

According to an embodiment of the invention, the present compositions are formulated as water dispersible granules.

Inventors of the present invention noted the ease of making compositions using Pyroxasulfone produced according to present invention.

According to an embodiment, the compositions of Pyroxasulfone obtained according to present invention are capable of dispersing quickly in water. According to an embodiment the compositions of Pyroxasulfone obtained according to present invention leads to optimum suspensibility while dispersed in water.

According to an embodiment, the composition prepared is a water dispersible granule comprising Pyroxasulfone, at least one dispersing agent and at least one wetting agent.

According to an embodiment, the dispersing agent/ wetting agent used is selected from, but not limited to, group comprising of anionic, cationic or zwitterionic and/or non-ionic surface-active compounds (surfactants) or combinations thereof, preferably anionic surfactant is used.

Examples of anionic surfactants include: anionic derivatives of fatty alcohols having 10-24 carbon atoms in the form of ether carboxylates, sulfonates, sulfates, and phosphates, and their inorganic salts (e.g., alkali metal and alkaline earth metal salts) and organic salts (e.g., salts based on amine or alkanolamine); anionic derivatives of copolymers consisting of EO (ethylene oxide), PO (propylene oxide) and/or BO (butylene oxide) units, in the form of ether carboxylates, sulfonates, sulfates, and phosphates, and their inorganic salts (e.g., alkali metal and alkaline earth metal salts) and organic salts (e.g., salts based on amine or alkanolamine) or acrylic/styrene copolymers, methacrylic copolymers; linear (C8-C15) alcohol derivative and their salts; alkyl aryl sulfonates including but not limited to alkyl benzenesulfonates; alkyl naphthalene sulfonates and salts thereof and salts of ligninsulfonic acid; derivatives of alkylene oxide adducts of alcohols, in the form of ether carboxylates, sulfonates, sulfates and phosphates, and their inorganic salts (e.g., alkali metal and alkaline earth metal salts) and organic salts (e.g., salts based on amine or alkanolamine); anionic derivatives of fatty acid alkoxylates, in the form of ether carboxylates, sulfonates, sulfates and phosphates, and their inorganic salts (e.g., alkali metal and alkaline earth metal salts) and organic salts (e.g., salts based on amine or alkanolamine); alkyl ether phosphate, alkyl sulfosuccinate mono ester and diester salts.

Preferably, sulfosuccinates and their derivatives/salts; acrylic/styrene copolymers; salts of lignin sulfonic acid are used.

According to an embodiment, the composition may further comprise a defoamer. The defoamer used is selected from, but not limited to, group comprising of aqueous emulsion with polysiloxane and emulsifier, silicone oil and magnesium stearate or a suitable combination thereof.

According to an embodiment, the water dispersible granule comprising Pyroxasulfone is prepared by a process comprising:
a) mixing Pyroxasulfone with wetting agents and dispersing agent/s as required;
b) milling the mixture in a suitable equipment to obtain a powder having a particle size D90≤15 μm; and
c) granulating the powder by suitable means and drying the granules obtained.

The inventors of the present invention have skilfully selected the range of temperature which ensures completion of the reaction for obtaining Pyroxasulfone of formula (I) substantially free of monoxide compound of formula (III). This finding of the present invention is based on multiple experimentation and vast research of inventor. During development of the process the major struggle was to remove the monoxide impurity from the Pyroxasulfone technical product. The process of present invention not only solves this problem of prior art but also improvises the yield of the process substantially.

### EXAMPLES

Analytical Method:
HPLC Method
Column-Zorbax SB C-8 (250 mm×4.6 mm, 5μ)
UV-Wavelength-220 nm
Column temperature-40° C.
Run time-30 min

The following examples are presented to provide what is believed to be the most useful and readily understood description of procedures and conceptual aspects of this invention. The examples provided below are merely illustrative of the invention and are not intended to limit the same to disclosed embodiments. Variations and changes obvious to one skilled in the art are intended to be within the scope and nature of the invention.

### Example 1: Preparation of Pyroxasulfone

To 31.76 g of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole was added 166 g of acetic acid, 1.28 g of sodium tungstate dihydrate and 22.24 g of 50% hydrogen peroxide. The mixture was stirred at 25-35° C. Then the temperature was increased to 50-55° C. and maintained for another 7 hours. The reaction was monitored by HPLC. After completion of reaction, the mixture was cooled to 25-30° C. and diluted with 48 g of water. The reaction mixture was then cooled to 0° C. and maintained for 1 hour. The product was filtered out washed with water and petroleum ether and dried to yield 30.3 g of Pyroxasulfone (Yield: 87.62%; Purity: 99.46% A/A; Monoxide impurity: Not detected in HPLC).

### Comparative Example 1: Preparation of Pyroxasulfone

2.8 g of 3-({[5-(difluoromethoxy)-1-methyl-3-(trifluoromethyl)-1H-pyrazol-4-yl]methyl}sulfanyl)-5,5-dimethyl-4,5-dihydro-1,2-oxazole was dissolved in 8 ml of acetic acid, and 80 mg of sodium tungstate dihydrate were added. Hydrogen peroxide (30%, 2.21 g, 20 mmol) was added dropwise at 23-34° C. within 20 min and the mixture was stirred at room temperature for 16 hours. The product was precipitated by adding 4 g of water and cooling to 1° C. After one hour at 10° C., the solid was filtered off and washed twice with 20 g of water and 20 ml of petroleum ether and dried to give 1.3 g of Pyroxasulfone (Yield: 43%) Monoxide impurity: 3.32% A/A.

This example is a reproduction experiment of Example 9C described in US20120264947.

Comparison of present invention and comparative example 1 is provided in Table No. 1 for better demonstration of the advantages of present invention and is not intended to limit the scope of invention in any way.

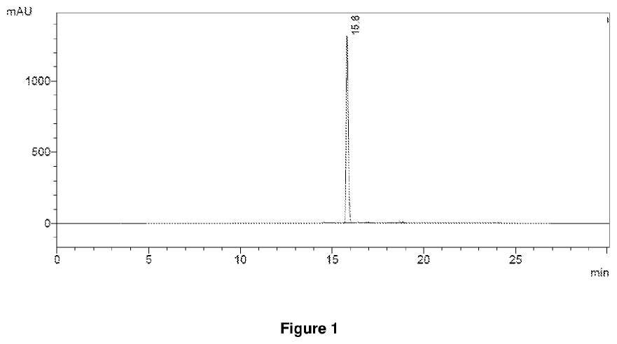

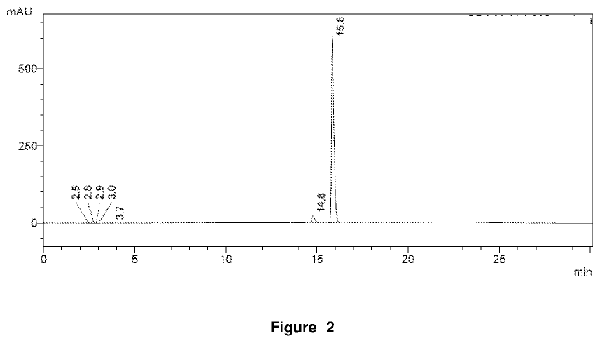

TABLE 1

| | Present | | Comparative | |
|---|---|---|---|---|
| | Invention | | Example 1 | |
| 3-({[5-(difluoromethoxy)-1- | 1 | mole | 1 | mole |
| methyl-3-(trifluoromethyl)-1H- | | | | |
| pyrazol-4-yl]methyl}sulfanyl)- | | | | |
| 5,5-dimethyl-4,5-dihydro-1,2- | | | | |
| oxazole | | | | |
| Sodium tungstate dihydrate | 0.001 to 0.1 | moles | 0.029 | moles |
| Acetic acid | 20 to 50 | moles | 17.94 | moles |
| Hydrogen peroxide | 2.6 to 5 | moles | 2.56 | moles |

| | | |
|---|---|---|
| Temperature at which reaction is | 50-55° C. | Room |
| carried out | | temperature |
| Pyroxasulfone (Yield) | >80% | 43% |
| HPLC Purity of Pyroxasulfone | Above 99% A/A | 96.52% A/A |
| Monoxide impurity i.e. compound | Not Detected | 3.32% A/A |
| of formula (III) | (For example | R.T - 14.8 |
| | refer to FIG. 1) | (Refer FIG. 2) |

The above table.1 shows the efficiency of the present invention process in removing the monoxide impurity to the great extent and improvising the yield of final product at least by 20% as compared to process known in prior art. The increase in mole ratio of acetic acid and the change in reaction condition have played main role in controlling the formation of undesired monoxide impurity.

### Example 2: Preparation of Pyroxasulfone 85% water dispersible granules Pyroxasulfone 85% Water dispersible granules (WDG) was prepared as follows:

| Sr. No. | Composition | Quantity (% w/w) |
|---|---|---|
| 1 | Pyroxasulfone | 86.8 |
| 2 | Wetting agent | 6 |
| 3 | Defoamer | 0.2 |
| 4 | Dispersing agent | 7 |
| | Total | 100 |

Pyroxasulfone along with wetting agent/s and dispersing agent/s were taken in ribbon blender and blended for 30 minutes. After blending, the powder was milled in air jet mill to achieve milled powder having particle size D90≤15 μm. The milled powder was then post blended in ribbon blender to form homogenous mixture. This mixture and required amount of Defoamer water solution (15 to 20%) were taken in dough maker to make dough suitable for extrusion. The dough was then extruded using extruder such as basket extruder by using required aperture size of 0.5 to 0.8 mm. The extruded granules were dried in fluid bed dryer to reduce moisture content below 2% and then sieved to get final product. The final product was characterised by X-ray powder diffraction pattern.

### Example 3: Preparation of Water Dispersible Granules Comprising Pyroxasulfone+Metribuzin

Pyroxasulfone+Metribuzin Water dispersible granules (WDG) was prepared as follows:

| Sr. No. | Composition | Quantity (% w/w) |
|---|---|---|
| 1 | Pyroxasulfone | 26.4 |
| 2 | Metribuzin | 44.0 |
| 3 | Wetting agent | 3 |
| 4 | Dispersing agent | 9 |
| 5 | Filler | q.s |
| | Total | 100 |

### Example 4: Preparation of Water Dispersible Granules Comprising Pyroxasulfone+Metribuzin

Pyroxasulfone+Metribuzin Water dispersible granules (WDG) was prepared as follows:

| Sr. No. | Composition | Quantity (% w/w) |
|---|---|---|
| 1 | Pyroxasulfone | 13.2 |
| 2 | Metribuzin | 22.0 |
| 3 | Wetting agent | 3 |
| 4 | Dispersing agent | 9 |
| 5 | Filler | q.s |
| | Total | 100 |

Having described what is considered the best form presently contemplated for embodying the present invention, various alterations, modifications, and/or alternative applications of the invention will be promptly apparent to those skilled in the art. Therefore, it is to be understood that the present invention is not limited to the practical aspects of the actual preferred embodiments hereby described and that any such modifications and variations must be considered as being within the spirit and the scope of the invention, as described in the above description.

1. A process for preparation of a pyroxasulfone of formula (I) substantially free of a monoxide compound of formula (III) 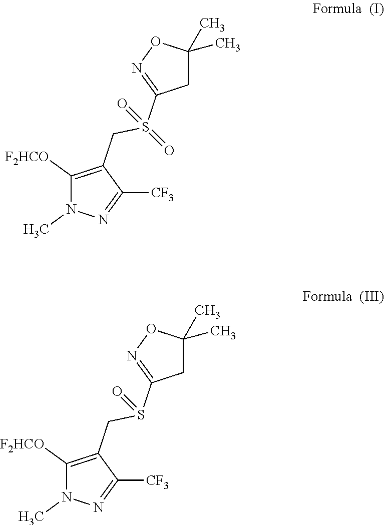 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]
said process comprising:
oxidizing a compound of formula (II) at a temperature ranging from 40 to 80° C.

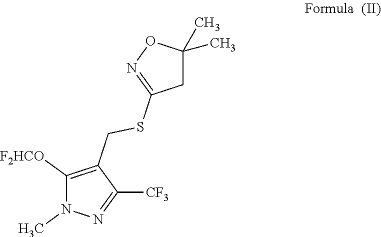 [SMILES: Cn1nc(C(F)(F)F)c(CSC2=NOC(C)(C)C2)c1OC(F)F]

2. The process as claimed in claim 1, wherein said compound of formula (II) is oxidized using an oxidizing agent.

3. The process as claimed in claim 2, wherein said oxidizing agent is selected from an organic peroxide and an inorganic peroxide.

4. The process as claimed in claim 3, wherein said oxidizing agent is hydrogen peroxide.

5. The process as claimed in claim 3, wherein the amount of oxidizing agent used is in the range of 2.6 to 5 moles with respect to the compound of formula (II).

6. The process as claimed in claim 1, wherein said compound of formula (II) is oxidized in the presence of a metal catalyst.

7. The process as claimed in claim 1, wherein said metal catalyst is selected from a tungsten catalyst, molybdenum catalyst, titanium catalyst, zirconium catalyst, and mixtures thereof.

8. The process as claimed in claim 7, wherein said metal catalyst is sodium tungstate or its dihydrate.

9. The process as claimed in claim 1, wherein said compound of formula (II) is oxidized in an organic acid solvent.

10. The process as claimed in claim 9, wherein said organic acid solvent is acetic acid.

11. The process as claimed in claim 9, wherein the amount of said organic acid solvent used is in the range of 20 to 50 moles with respect to the compound of formula (II).

12. A process for the preparation of a pyroxasulfone of formula (I) substantially free of a monoxide compound of formula (III) 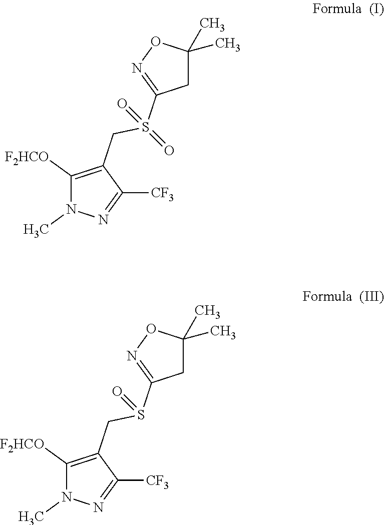 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]
said process comprising:
oxidizing a compound of formula (II) in the presence of an oxidizing agent and a metal catalyst at a temperature ranging from 40 to 80° C. in acetic acid, 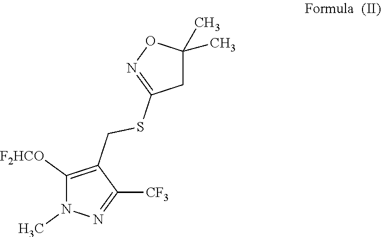 [SMILES: Cn1nc(C(F)(F)F)c(CSC2=NOC(C)(C)C2)c1OC(F)F]
wherein the amount of acetic acid used is in the range of 20 to 50 moles with respect to the compound of formula (II).

13. A pyroxasulfone of formula (I) substantially free of a monoxide compound of formula (III)

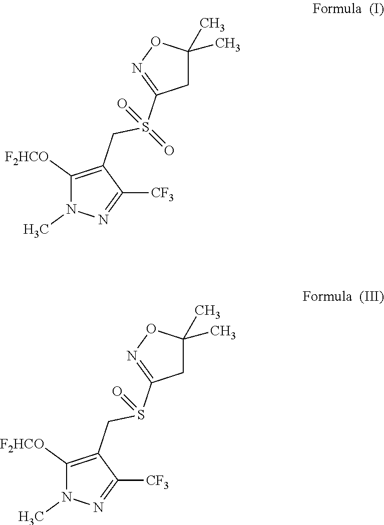 [SMILES: Cn1nc(C(F)(F)F)c(CS(=O)(=O)C2=NOC(C)(C)C2)c1OC(F)F ; Cn1nc(C(F)(F)F)c(CS(=O)C2=NOC(C)(C)C2)c1OC(F)F]

14. An agrochemical composition comprising pyroxasulfone of formula (I) as claimed in claim 13.

15. The agrochemical composition as claimed in claim 14, wherein said composition comprises at least one triazinone herbicide and an agrochemically acceptable excipients.

16. The agrochemical composition as claimed in claim 15, wherein said composition is formulated as water dispersible granules.
